(19) United States
(12) Patent Application Publication
(10) Pub. No.: US 2016/0213236 A1
(43) Pub. Date: Jul. 28, 2016

(54) VISUALIZATION INSTRUMENT

(71) Applicant: KING SYSTEMS CORPORATION, Noblesville, IN (US)

(72) Inventors: Amy Hruska, Indianapolis, IN (US); Zachary Wagner, Noblesville, IN (US); Matt Streicher, Indianapolis, IN (US); Dennis C. Leiner, Portland, ME (US); Bryan E. Rolfs, Chicago, IL (US)

(21) Appl. No.: 14/914,595

(22) PCT Filed: Aug. 26, 2014
(86) PCT No.: PCT/US2014/052780

§ 371 Date: Feb. 25, 2016

Related U.S. Application Data
(60) Provisional application No. 61/870,076, filed on Aug. 26, 2013.

(51) Int. Cl. A61B 1/05; H04N 5/225; A61B 1/00

(52) U.S. Cl. CPC A61B 1/05; A61B 1/00195; A61B 1/0011; H04N 5/2252; H04N 5/2256; H04N 5/2254; H04N 2005/2255

(57) ABSTRACT

A visualization instrument comprising a camera including an optical train and an image sensor. The optical train includes at least one lens and may include a prism. Optical images received by the optical train are captured by the image sensor, which may be positioned adjacent the image sensor to reduce the profile of the camera.

### CROSS-REFERENCE TO RELATED APPLICATIONS

This application claims the benefit of priority from U.S. Provisional Patent Application No. 61/870,076, filed Aug. 26, 2013. The disclosure of said patent application is expressly incorporated herein by reference in its entirety.

### FIELD OF THE DISCLOSURE

The present disclosure relates generally to a visualization instrument including a camera communicatively coupled with a display device. More specifically, the present disclosure relates to a visualization instrument including a camera insertable into an internal space.

### BACKGROUND OF THE DISCLOSURE

Visualization instruments include medical and non-medical instruments. Medical visualization instruments are used in a multitude of procedures including laryngoscopy, colonoscopy, rhinoscopy, bronchoscopy, cystoscopy, hysteroscopy, laparoscopy, arthroscopy, etc. Generally, a medical visualization instrument comprises a camera and structure arranged to support the camera during the procedure. The structure may be configured for the particular procedure, and the instrument may thus be given a name corresponding to the procedure. Exemplary instruments include laryngoscopes, bronchoscopes, endoscopes etc. Non-medical visualization instruments are used to investigate the internal structures of machines, buildings, and explosive devices, for example.

Laryngoscopes provide views of the vocal folds and the glottis after the laryngoscope has been inserted into the buccal cavity of the patient. Direct laryngoscopy is usually carried out with the patient lying on his or her back. During direct laryngoscopy, the laryngoscope is inserted into the mouth, typically on the right side, and pushed towards the left side to move the tongue out of the line of sight and to create a pathway for insertion of an endotracheal tube. The blade may be lifted with an upward and forward motion to move the epiglottis and make a view of the glottis possible. Once the laryngoscope is in place, the endotracheal tube may be inserted into the pathway. The blade may be provided with guide surfaces to guide the insertion of the endotracheal tube.

Laryngoscopes may be outfitted with optical devices to provide views of the vocal cords externally of the patient's body. Optical devices include lenses, mirrors and fiberoptic fibers, all adapted to transfer an optical image. Devices may also be provided to capture the optical images and display corresponding images in video display screens and/or monitors.

Traditional visualization instruments have limitations such as, for example, fogging, insufficient lighting to produce a good optical image, inability to project images remotely, additional procedural steps to insert the endotracheal tube, and cost, to name a few. Further, there is a need to reduce the size of the camera to reduce the invasiveness of medical procedures and for pediatric care.

### SUMMARY OF EMBODIMENTS OF THE DISCLOSURE

A visualization instrument and a method of making the visualization instrument are disclosed herein. In an exemplary embodiment, the visualization instrument is a video laryngoscope. In another exemplary embodiment, the visualization instrument is configured for non-medical uses. In embodiments of the visualization instrument, the visualization instrument includes a camera. The camera includes a light source configured to illuminate structures in a target space; an image sensor having an imaging surface; and an optical train including one or more lenses and a prism. The optical train is configured to receive light reflected from the illuminated structures and refract optical images of the illuminated structures to the image sensor. The image sensor generates an image stream including images corresponding to the optical images. The camera also includes a support structure supporting the light source, the image sensor and the optical train; and a housing enclosing the support structure, the light source, the image sensor and the optical train.

In embodiments of the disclosure, a visualization instrument is disclosed, the visualization instrument comprising a camera. The camera includes a light source adapted to illuminate structures in a target space; a support structure; a flat cable; an image sensor electrically coupled to the flat cable; and an optical train including one or more lenses and a prism. The prism is located adjacent the image sensor. The image sensor generates an image stream. The optical train is sealed in the support structure. The instrument further comprises an assembly housing enclosing the support structure, the light source, the image sensor, and the optical train.

In embodiments of the disclosure, a method of making a visualization instrument is disclosed, the method comprising: electrically coupling an image sensor to a flat cable; mounting a light source on a support housing; and inserting an optical train into the support housing. The optical train includes lenses and a prism. The prism is located adjacent the image sensor after the optical train is inserted into the support housing. The method further comprises sealing the optical train in the support housing; and enclosing the support housing with an assembly housing.

In embodiments of the disclosure, the image sensor is adhered to the prism. The image sensor may be adhered to the prism before the optical train is inserted in the support housing.

In embodiments of the disclosure, the instrument includes a circuit configured to reorient an image stream output by the image sensor such that the reoriented image stream matches the orientation of the structures illuminated by the light source. In one example, the circuit comprises an orientation processor.

The features of this invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIGS. 1 and 2 are posterior and lateral plan views of an embodiment of a video laryngoscope set forth in the disclosure;

FIG. 3 is a plan view of the distal end of the video laryngoscope of FIGS. 1 and 2;

FIG. 4 is a perspective view of an embodiment of a camera set forth in the disclosure;

FIGS. 5 and 6 are cross-sectional views of embodiments of cameras set forth in the disclosure;

FIGS. 7 and 8 are schematic representations corresponding to further embodiments of cameras set forth in the disclosure;

FIGS. 9 and 10 are partial perspective and exploded views of a further embodiment of a camera set forth in the disclosure;

FIGS. 11, 12 and 13 are plan views of embodiments of optical components of cameras set forth in the disclosure;

FIG. 14 is an exploded view of an additional embodiment of a camera set forth in the disclosure;

FIGS. 15, 16 and 17 are block diagrams of embodiments of electronic components of cameras set forth in the disclosure;

FIGS. 18 and 19 are plan and perspective views of an image presentation component removably connected to cameras according to embodiments of methods set forth in the disclosure;

FIGS. 20 and 21 are lateral plan views of an embodiment of a camera adapter set forth in the disclosure; and

FIGS. 22 and 23 are perspective views of another embodiment of a camera adapter set forth in the disclosure.

Corresponding reference characters indicate corresponding parts throughout the several views. Although the drawings represent embodiments of the present invention, the drawings are not necessarily to scale and certain features may be exaggerated to better illustrate and explain the embodiments. The exemplifications set out herein illustrate embodiments of the invention in several forms and such exemplification is not to be construed as limiting the scope of the invention in any manner.

### DETAILED DESCRIPTION

The embodiments of the disclosure discussed below are not intended to be exhaustive or limit the invention to the precise forms disclosed in the following detailed description. Rather, the embodiments are chosen and described so that others skilled in the art may utilize their teachings.

Generally, in embodiments of a camera set forth herein, the camera includes an optical train and an image sensor having an imaging surface. The optical train includes two or more lenses aligned to form a line of sight of the camera. The optical train may also include a prism configured to guide the line of sight to the imaging surface. The optical train receives light reflected from an object in a target space, and the image sensor generates a corresponding electronic image or video for presentation with a display screen. The prism changes the orientation of the image stream reflected by the lenses to enable construction of a camera with reduced cross-sectional area. The electronic images are communicated to devices that may display the electronic images or may reformat the electronic images before they are displayed.

Since the intrusiveness of a medical procedure may be determined by the size of the camera, reducing the cross-sectional area of the camera may enable performance of comparably less intrusive medical procedures or performance of procedures in pediatric patients. Similarly, a smaller camera may also enable use of visualization instruments in spaces smaller than previously possible.

Embodiments of a visualization instrument including the aforementioned camera and others, and embodiments of a method of using and making the visualization instrument, are also disclosed herein. The visualization instrument is insertable into a space to capture images of tissues or objects located in the space. While the embodiments of the disclosure are applicable in medical and non-medical applications, exemplary features of visualization instruments will be described below with reference to a video laryngoscope. It should be understood that the invention is not so limited. The features described below may be equally applicable to any medical and non-medical applications and instruments.

As stated above, the camera includes an optical train and an image sensor having an imaging surface. The optical train includes two or more lenses and a may include a prism. Light enters the camera through a camera view port and is refracted by the optical train to the image sensor. As used herein, a prism is a light-reflecting optical component bounded by two or more faces to change the direction of light travel. The prism includes an inlet face and an outlet face. Light may be refracted one or more times after it enters the prism through the inlet face before the light exits the prism through the outlet face. Exemplary prisms include a right-angle prism and a pentaprism. In the right-angle prism, the inlet and outlet faces are disposed at a 90 degree angle. The imaging surface is parallel and adjacent to the outlet face of the prism. Prisms may invert or flip (e.g. re-orient the image such that objects appear inverted) and/or mirror (e.g. re-orient the image such that its right side appears on the left side of the mirrored image) the optical image.

The image sensor comprises an integrated circuit with an imaging surface which is configured to generate the electronic images based on the light refracted by the prism. The area of the integrated circuit measured on a plane including the imaging surface is larger than the area of the imaging surface. For example, the imaging surface area may comprise 50% of the surface area of the integrated circuit. Therefore, if the image sensor is positioned parallel to the camera's view port, the smallest camera cross-section for a given image sensor may be determined by the size of the image sensor. If the image sensor is not positioned parallel to the camera's view port, then the cross-section of the camera may be reduced. Electronic circuits are provided to process the image stream to compensate for the non-parallel orientation of the image sensor relative to the camera's view port.

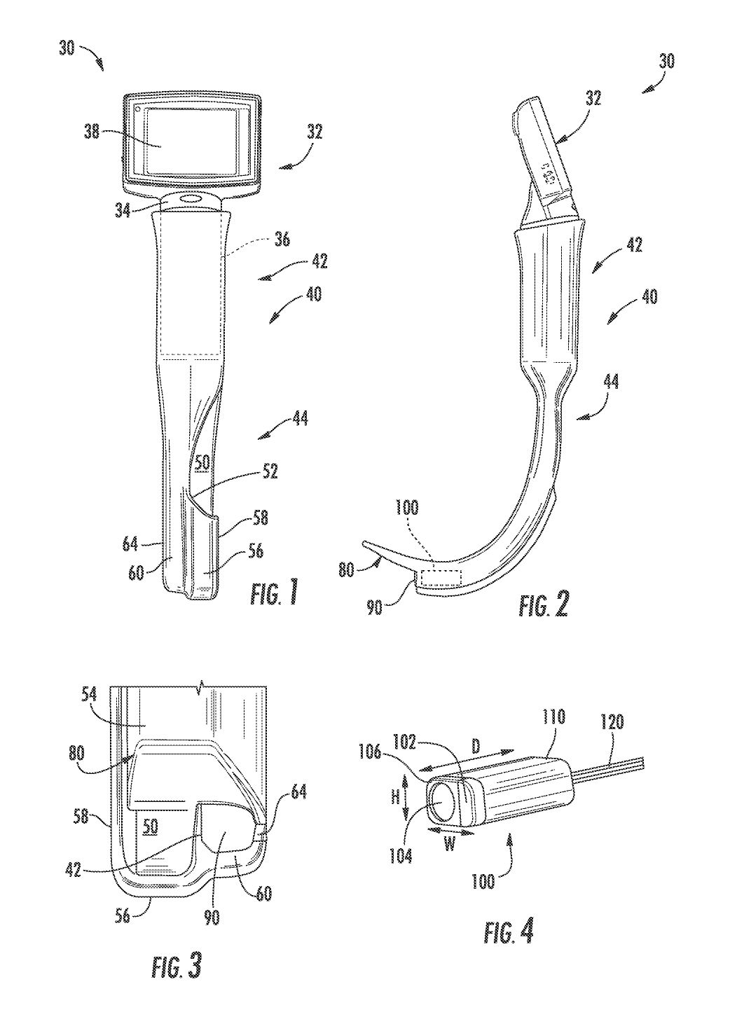

Referring to FIGS. 1, 2 and 3, an embodiment of a visualization instrument, denoted by numeral 30, comprises image presentation component 32 and a blade 40. Image presentation component 32 includes a display support structure 34, coupling a battery housing 36 and a display screen 38. Blade 40 includes a handle portion 42 integrally formed with an insertable portion 44. In the present embodiment, blade 40 is shown as a single part integrally combining handle portion 42 and insertable portion 44. In a variation thereof, the handle portion and the insertable portion are distinct parts that are removably attachable. Handle portion 42 comprises a proximal cavity configured to receive battery housing 36. When battery housing 36 is received in the proximal cavity, blade 40 supports image presentation component 32, forming a self-contained portable visualization instrument. A portable visualization instrument may be sized so that it can be hand-held. Blade 40 may be molded from polymeric materials.

As shown, insertable portion 44 comprises an elongate guide pathway 50 configured to facilitate insertion of an endotracheal tube, catherer and the like (not shown) into the larynx of a patient. Guide pathway 50 is positioned on one side of a medial wall 52. Guide pathway 50 is further defined by an anterior guide wall 54, a posterior guide wall 56, and a lateral guide wall 58 (positioned opposite medial wall 52). An electronics pathway (not shown) is positioned on the opposite side of medial wall 52, in a side-by-side arrangement. The electronics pathway is defined by medial wall 52, anterior wall 54, a posterior electronics pathway wall 60 and a lateral electronics pathway wall 64. The cross-sectional area of the electronics pathway may have square, circular or any other shape. In a variation of the present embodiment, insertable portion 44 does not include lateral guide wall 58 or posterior guide wall 56, and the guide pathway is formed by the surfaces of medial wall 52 and anterior wall 54. The electronics pathway extends from a proximal end of insertable portion 44 to a blade view port 90 located at the distal end of insertable portion 44. A camera 100, shown in FIG. 4, is positioned at the distal end of the electronics pathway such that camera 100 illuminates the space in front of the distal end of insertable portion 44 (e.g. the target space) to capture images of tissues or an object positioned therein. In both variations of the present embodiment, anterior wall 54 extends beyond blade view port 90 to form a truncated tip 80, which is configured to lift the epiglottis. A translucent view port cover may be sealably attached to blade view port 90 to seal the electronics pathway from moisture and dirt.

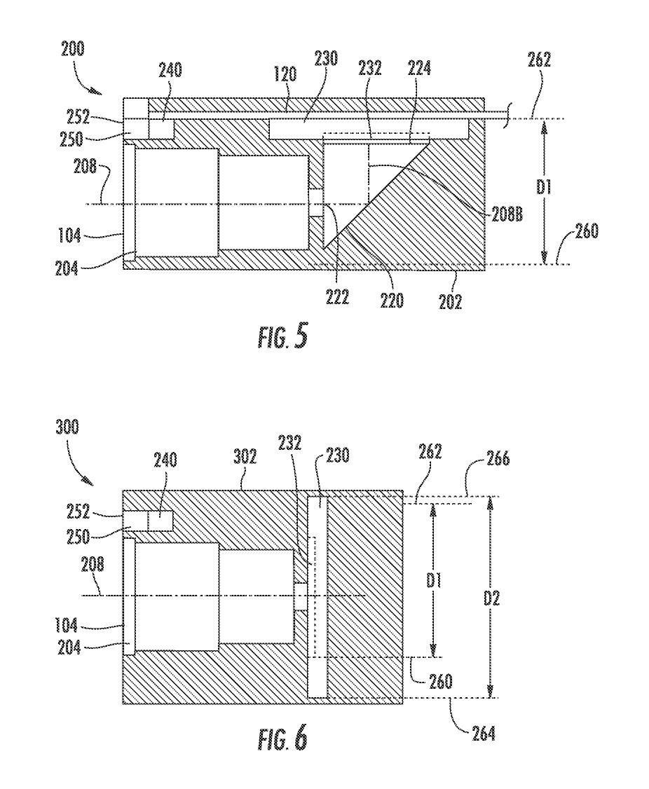

Referring to FIG. 4, camera 100 comprises an LED cover 102, a support structure 106, an assembly housing 110 and a wire bundle 120. Exemplary wire bundles include flat cables, ribbon cables, a cable including the bundle of wires, and a bundle of individual wires, and any other configuration of wires. Camera 100 also comprises an optical train including a prism (not show). Support structure 106 includes a camera view port 104 and is configured to support an LED 240 (shown in FIG. 5) and an image sensor 230 (shown in FIG. 5). LED 240 illuminates the target space and image sensor 230 captures images of illuminated tissues and/or objects therein. Wire bundle 120 is a conduit for the transfer of power, control signals and the video stream between LED 240, image sensor 230, and image presentation component 32. Alternative, a wireless conduit for the transfer of one or more of power, control signals and the video stream may be provided. Exemplary wireless conduits are disclosed in commonly owned U.S. patent application Ser. No. 13/941,183, filed Jul. 12, 2013, which is incorporated by reference herein in its entirety.

LED cover 102 is translucent and encloses LED 240. LED cover 102 is attached to support structure 106. Assembly housing 110 encloses support structure 106. LED 240 and image sensor 230 may be potted within assembly housing 110. Assembly housing 110 may be made of a robust material to protect LED 240 and image sensor 230. Exemplary robust materials include aluminium, copper, steel, and polymers, which may include glass or carbon fiber reinforcements. Assembly housing 110 may comprise a conductive metal to reduce electromagnetic interference. It should be understood that LED 240 may comprise any number of shapes, forms and power requirements, and that references to LED 240 in various embodiments herein is not intended to suggest that the particular LEDs used in each embodiment have the same shape, form or power requirement. More generally, any light source may be used instead of LED 240. Exemplary light sources include filament lamps and light guides comprising fibers.

The height, width and depth of camera 100 are denoted by the letters H, W and D. As used herein, the height and width of the cameras are intended to describe two substantially orthogonal dimensions of the cameras which do not necessarily correspond to the height and width of the electronics pathway. As shown in FIG. 4, the height of camera 100 corresponds to the anterior/posterior height of the blade, defined by the distance between the anterior and posterior guide walls, at the distal end of the blade. The width of the insertable portion includes the width of the guide pathway and the width of the electronics pathway, which is dimensioned to encompass the width of camera 100. In another variation (not shown), camera 100 is rotated relative to the orientation shown in FIG. 4, such that its width dimension is about parallel to the anterior/posterior height of the distal end of the blade. Camera 100 may be rotated relative to the anterior/posterior height by any amount.

Additional cameras 200, 300 and 400, described with reference to FIGS. 5, 6 and 10 describe different arrangements of components of the respective cameras. All the cameras include a light source, exemplified as LED 240 and an image sensor. The cameras present exemplary constructions configured to reduce the dimensions of the respective camera. The cameras may enable operation of multiple blade configurations with a common display support structure. With a common display support structure, multiple blades may be provided in a kit, such as an emergency response kit. Exemplary blades include adult and pediatric blades, and channeled and channelless blades (e.g. blades with and without posterior and lateral guide walls). A kit may also include a stylet, an endoscope and a snake-cam (a malleable wire harness with a camera) and any other device configured to operate with image presentation component 32.

In one variation of the present embodiment, wire bundle 120 is affixed to image presentation component 32. In the present variation, when image presentation component 32 is coupled to the handle cavity, wire bundle 120 and camera 100 are positioned in the electronics pathway. When image presentation component 32 is removed from the handle cavity, wire bundle 120 and camera 100 are removed from the electronics pathway. Wire bundle 120 may be permanently or removably affixed to image presentation component 32, in both cases removably positionable in the electronics pathway. In another variation of the present embodiment, wire bundle 120 is permanently affixed to the electronics pathway and removably coupled to image presentation component 32. In both variations, the size of blade view port 90, and of the electronics pathway, can be reduced if the size of camera 100 is reduced. Thus, reducing the size of camera 100 may facilitate less intrusive medical procedures.

FIG. 5 is a cross-sectional view of an embodiment of a camera denoted by numeral 200. Camera 200 comprises a support structure 202 including camera view port 104 and a lens cavity 204 configured to receive lenses forming, in part, an optical train having an optical centerline 208. Camera 200 also comprises a right-angle prism 220 including an inlet face 222 and an outlet face 224. Prism 220 redirects light received by inlet face 222 along camera centerline 208 toward outlet face 224 along centerline 208B, which intersects imaging area 232 of image sensor 230. Redirection of the light by prism 220 enables placement of image sensor 230 substantially perpendicular to camera view port 104. Camera 200 further comprises an LED cavity 250 including an illumination port 252 through which light from LED 240 emanates to the target space.

As shown in FIG. 5, camera 200 is rotated 90 degrees relative to the posture of camera 100, shown in FIG. 4, to better illustrate the arrangement of its component parts. FIG. 5 also shows first and second planes 260 and 262, spaced by a distance D1. First plane 260 extends parallel to the furthest point of camera view port 104 away from second plane 262, and second plane 262 extends parallel to the non-sensing surface of image sensor 230. Thus, D1 represents the smallest distance, along a plane parallel to camera view port 104, which encompasses projections of image sensor 230, LED 240 and camera view port 104, in their entirety. Distance D1 thus corresponds to the smallest possible width of camera 200, such that increasing D1 increases the width of camera 200. Of course, if camera 200 were positioned in the blade as shown in FIG. 5 (with the LED aligned in the anterior/posterior direction with the camera view port), then distance D1 would represent the smallest possible height of camera 200.

Camera 200 also comprises wire bundle 120. In the present embodiment, wire bundle 120 is shown as a flat cable which includes a ball grid array (not shown) configured to couple the flat cable with image sensor 230. Wire bundle 120 also provides power to LED 240. In the present embodiment, LED 240 is positioned between wire bundle 120 and camera 200 beneath second plane 262, thereby preserving the smallest possible width (or height) of camera 200. As LED 240 is moved away from camera view port 104 or increased in size, the smallest possible width (or height) of camera 200 would increase accordingly. Thus, positioning an LED at least partially in front of an image sensor, so that a projection of the LED at least partially overlaps the image sensor (when viewed from the camera view port), further reduces or preserves the small cross-sectional area of the camera.

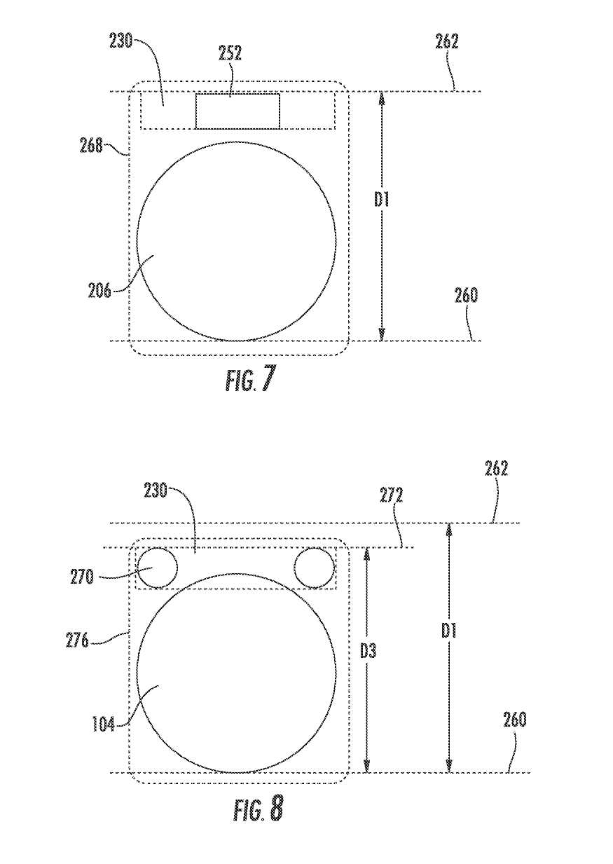

FIG. 6 is a cross-sectional view of a conventional camera, denoted by numeral 300, comprising a support structure 302 supporting image sensor 230 and LED 240. Camera 300 does not include right-angle prism 220. A distance D2 between planes 264 and 266 represents the smallest distance, along a plane parallel to camera view port 104, which encompasses projections of image sensor 230, LED 240 and camera view port 104, in their entirety. Due to the absence of right-angle prism 220, image sensor 230 is parallel to camera view port 104 and, due to the length and width of image sensor 230 being larger than its thickness, D2 is larger than D1.

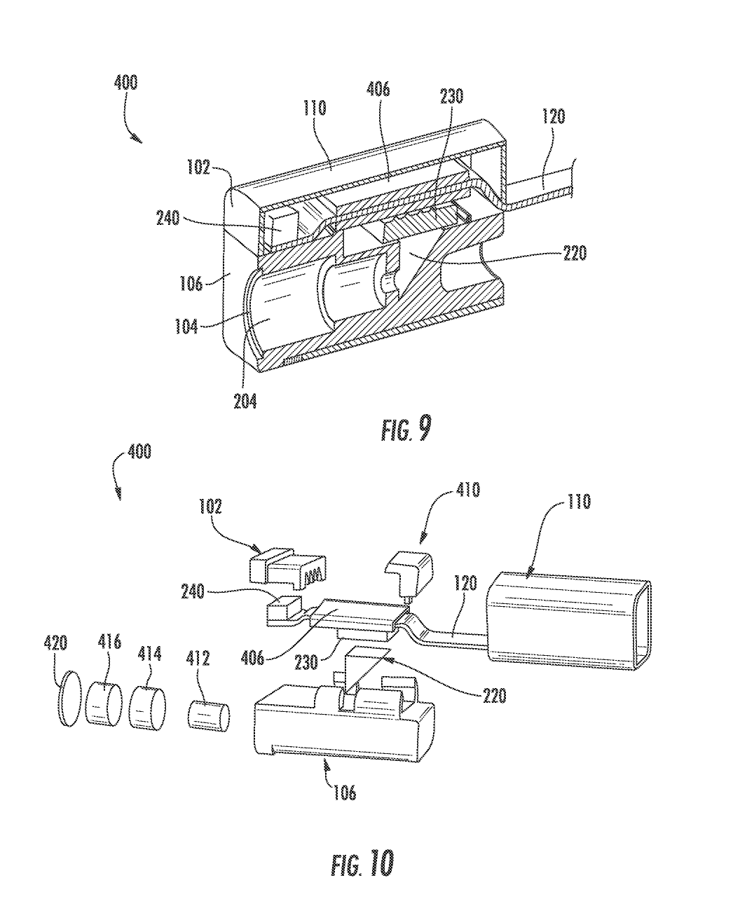

FIGS. 7 and 8 are schematic representations of further embodiments of cameras. FIG. 7 is a view of illumination port 252, image sensor 230, and camera view port 104 as seen from the viewing end of camera 200. A cross-sectional area 268, or profile, of camera 200 is also shown. FIG. 8 is a view of a variation of camera 200 in which LED 240 is substituted by two round LEDs. Two illumination ports 270 are shown. Also shown is a cross-sectional area 276 or profile of the resulting camera. The profile and position of image sensor 230 controls in part the profile of the camera. By lowering image sensor 230 and radially offsetting the LEDs and illumination ports 270, the cross-sectional area 276 is reduced relative to the cross-sectional area 268, which reduction is also represented by a smaller dimension D3 as compared to D1. In the embodiment shown, camera view port 104 is positioned at least partially in front of image sensor 230, so that a projection of camera view port 104 overlaps image sensor 230.

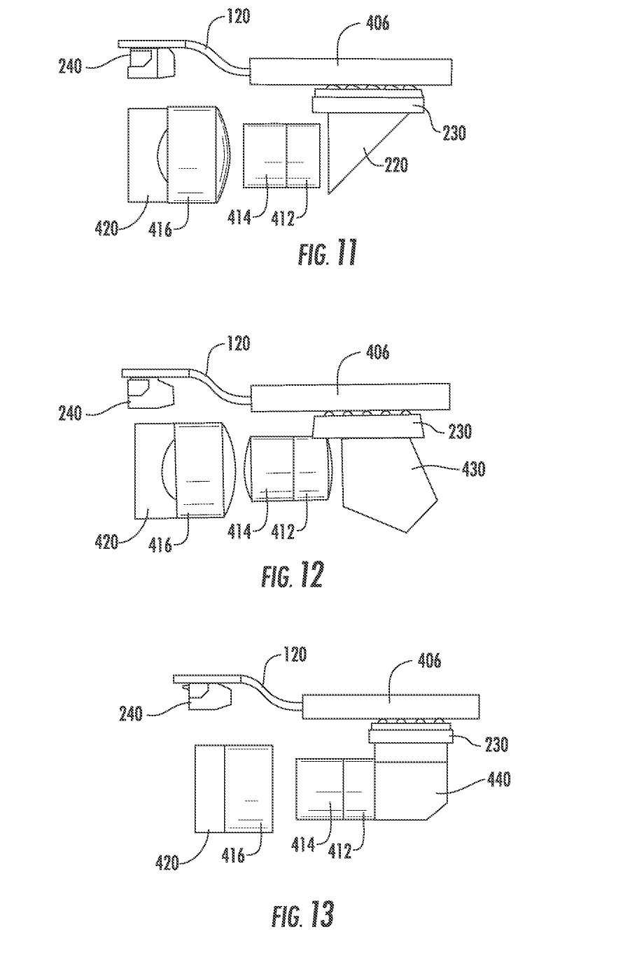

FIGS. 9 and 10 are partial perspective and exploded views of a further embodiment of a camera set forth in the disclosure, denoted by numeral 400. Camera 400 is similar to cameras 100 and 200. As in camera 200, camera 400 comprises support structure 106, that supports right-angle prism 220, image sensor 230 and LED 240. Camera 400 further comprises a circuit board 406 and electronic components mounted thereon. As shown in FIGS. 9 and 10, a circuit board is provided on each side of wire bundle 120. In one example, the electronic components are positioned between the circuit boards. Exemplary electronic components include a power regulator (voltage or current) and an orientation processor. A power regulator may be provided to convert power received from image presentation component 32 to a different form or level. The power regulator may convert an input voltage to an output voltage of a different voltage value, or to a constant current, for example. The power regulator may be provided to power the orientation processor. The orientation processor may be provided to cause the image sensor to change the orientation of the video stream to compensate for the orientation changes due to the use of a prism. Re-orientation may be desired to compatibilize different blades and camera support structures with a common image presentation component 32. If compatibilization is not desired, the image stream may be re-oriented by a processor in the image presentation component 32. Image presentation component 32 may convert the image stream output by image sensor 230 to a different size and transmit to resized image stream to a remote device. Remote devices include computers and portable communication devices. Portable communication devices include portable computers, smart phones and tablets.

Camera 400 also comprises lenses conveying light to prism 220. Exemplary lenses 412, 414 and 416 are shown. Also shown is a view port cover 420. In one example, view port cover 420 is sealingly attached to camera view port 104 to seal the optical train from moisture and dirt. In another example, view port cover 420 is omitted and an adhesive material is applied to the most distal lens, in this case lens 416, to seal the optical train. Unlike camera 200, wire bundle 120 extend between LED 240 and camera view port 104. A proximal support 410 cooperates with LED cover 102 to support assembly housing 110, which is slidably received over proximal support 410 and a portion of LED cover 102. In another example, proximal support 410 extends over circuit board 406. In a variation thereof, proximal support 410 extends toward and proximally of support structure 402 to provide a rear or proximal closure.

FIGS. 11, 12 and 13 are plan views of embodiments of cameras showing an exemplary optical trains including lenses 412, 414 and 416, and a prism. Exemplary prisms 220, 430 and 440 are shown, respectively, in FIGS. 11, 12 and 13. Prism 542 is a penta-prism.

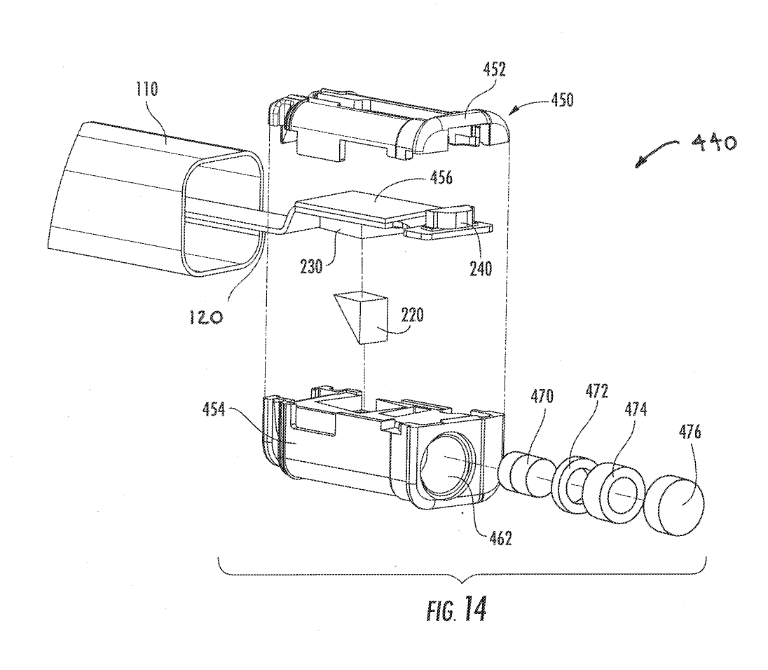

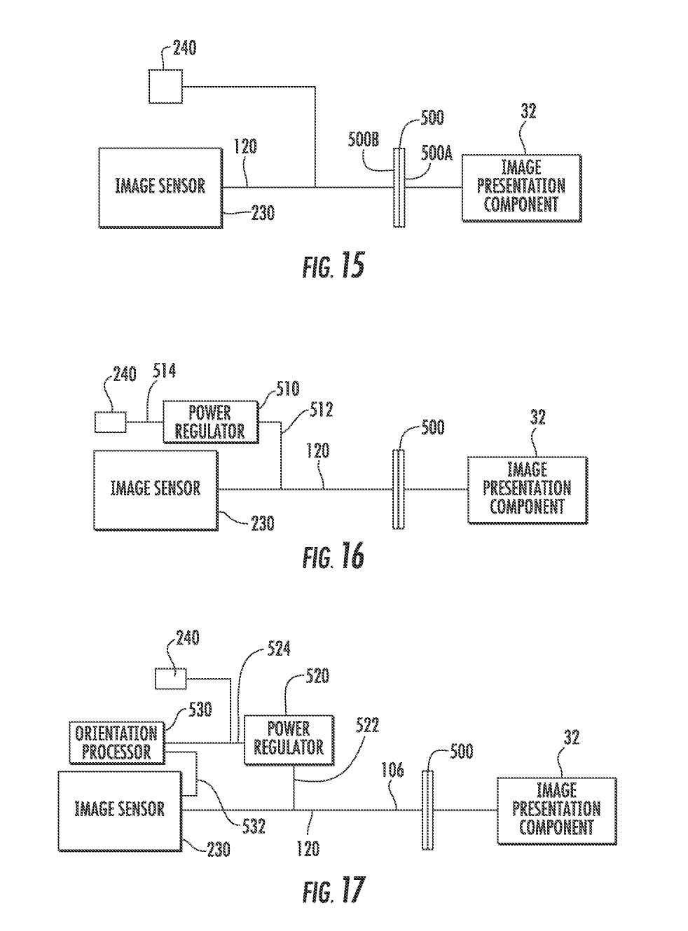

FIG. 14 is an exploded view of another embodiment of a camera, denoted by numeral 440. Camera 440 is similar to cameras 100 and 200. Camera 440 includes a two-part support structure 450 comprising first support structure 454 and second support structure 452. First and second support structures 454 and 452 are adapted to enclose prism 220, image sensor 230, and LED 240. Camera 440 further comprises lenses 470, 472, and 474, and may comprise a lens cover 476. In one example, view port cover 476 is sealingly attached to a view port 462 of first support structure 454 to seal the optical train from moisture and dirt. In another example, view port cover 476 is omitted and an adhesive material is applied to the most distal lens, in this case lens 474, to seal the optical train. The adhesive material may comprise an ultraviolet curable optical clear adhesive. A power regulator may be provided to convert power received from image presentation component 32 to a different form or level. The power regulator may convert an input voltage to an output voltage of a different voltage value, or to a constant current, for example. The power regulator may be provided to power the orientation processor. The orientation processor may be provided to cause the image sensor to change the orientation of the video stream to compensate for the orientation changes due to the use of a prism. Re-orientation may be desired to compatibilize different blades and camera support structures with a common image presentation component 32. If compatibilization is not desired, the image stream may be re-oriented by a processor in the image presentation component 32. Image presentation component 32 may convert the image stream output by image sensor 230 to a different size and transmit to resized image stream to a remote device.

A stiffener component 456 may be coupled to wire harness 120, illustratively a flexible flat cable, to ensure proper mounting of image sensor 230. Unlike camera 400, circuit board 406 has been removed to reduce the size of the camera. Electronic components may be mounted on connector 500B, as shown in FIG. 22 described further below. Small electronic components may be mounted on wire harness 120. In one example, integrated circuits are mounted on connector 500B and resistors, capacitors, and other passive components are mounted on wire harness 120. In one example, passive components are mounted opposite image sensor 130. LED 240 is supported by wire harness 120. In the present embodiment, second support structure 452 includes a distal cavity 460 and LED 240 is positioned in distal cavity 460, but second support structure 452 does not fully enclose LED 240. In one variation, an adhesive material is applied to LED 240 and second support structure 452 to seal LED 240 in support structure 450.

An advantage of camera 440 is reduced size. This is particularly desirable in pediatric medical devices where the amount of space available to perform procedures is much less than the space available in adult patients. Another advantage is ease of manufacture. In one embodiment of a method of making a camera, the method comprises: adhering an image sensor to a prism, electrically coupling the image sensor to a flat cable, inserting the prism into a prism cavity of a first support structure, inserting lenses into a lens cavity of the first support structure, the lenses including a proximal lens and a distal lens, the proximal lens located adjacent the prism, sealing the lenses in the lens cavity, and enclosing the first support structure with an assembly housing. In one example, sealing the lenses comprises adhering the distal lens to the first support structure. In another example, sealing the lenses comprises adhering a lens cover to the first support structure. In some variations, inserting the prism into a prism cavity is performed after the image sensor is adhered to the prism.

In one variation, the method further comprises coupling a second support structure to the first support structure before enclosing the first support structure with the assembly housing. In a further variation, wherein the first support structure and the second support structure form a housing structure, the method further comprises sealing an LED in the housing structure. In one example, sealing the LED comprises adhering the LED to the housing structure.

In one variation of the present embodiment, the assembly housing comprises metal. In one example, the assembly housing comprises assembly housing 110. In one example, assembly housing 110 is made of metal. In another example, the metal is stainless steel. In another variation, adhering of the prism and the image sensor is performed after the prism is inserted in the prism cavity. In another variation, a lens cover is adhered to a view port of the first support structure to seal the lenses therein. The components described herein may be the components of camera 440.

FIGS. 15, 16 and 17 are block diagrams of a visualization instrument including electronic components suitable for use with any camera described herein, including cameras 100, 200, 300, 400 and 440. The electronic components described below, such as power regulators and orientation processors, may be disposed on a camera, as described below, or may be disposed on a wire harness connecting the camera with the image presentation component. The wire harness may be coupled to the image presentation component with an electrical coupling. An optional electrical coupling 500 is shown in FIGS. 15, 16 and 17, comprising two connectors, one attached to image presentation component 32 (denoted as 500A) and the other attached to wire bundle 120 (denoted as 500B). In one example, wire bundle 120 extends from image sensor 230 and LED 240 to image presentation component 32 without electrical coupling 500. Thus, image sensor 230 and LED 240 are permanently connected to image presentation component 32. In another example, wire bundle 120 extends from image sensor 230 and LED 240 to image presentation component 32 through electrical coupling 500. In the present example, image sensor 230 and LED 240 are removably attachable to image presentation component 32 and may be permanently attached to a blade or to a blade adapter, such as blade 40 and blade adapter 600 (shown on FIG. 18). When the blade or blade adapter is detached from image presentation component 32, image presentation component 32 is disconnected from the camera.

FIG. 16 illustrates an optional power regulator 510 receiving power from image presentation assembly 32 and providing power to LED 240. By providing a power regulator in the camera, different LED configurations can be designed to satisfy different lighting requirements from the same power source. For example, one blade may receive a constant current from image presentation component 32, and another blade may convert the constant current (received via a conductor 512) to a different level of constant current suitable to provide a different light intensity or meet the rated requirements of a differently sized LED (supplied to the LED via conductor 514). In another example, a lamp is used instead of an LED, and the power regulator converts the power received from image presentation component 32 to a different level (higher or lower) or type (voltage to current or current to voltage). Further, the intensity of the LED may be controlled by a control signal from image presentation component 32 which changes a feedback loop coupled to the power regulator (in which case line 512 represents a power conductor and also a control signal). For example, a feedback voltage or current may be changed by switching a transistor on or off to change a resistance in the feedback look, which resistance controls the output voltage or current of the power regulator (e.g. a switching regulator). Feedback loops used with power regulators are known.

FIG. 17 illustrates an optional orientation processor 530 coupled to wire bundle 120 and to image sensor 230. Use of an orientation processor facilitates use of different optical trains with a common image presentation component, without modification of the image presentation component. Alternatively, image presentation component may include video processing logic operable to re-orient the video stream if necessary. The term “logic” as used herein includes software and/or firmware executing on one or more programmable processors, application-specific integrated circuits, field-programmable gate arrays, digital signal processors, hardwired logic, or combinations thereof. Therefore, in accordance with the embodiments, various logic may be implemented in any appropriate fashion and would remain in accordance with the embodiments herein disclosed.

Any processor of small enough size is suitable for use as orientation processor 530. Exemplary processors include microcontrollers such as AVR flash microcontrollers marketed by Atmel Corporation under the designation tinyAVR (e.g. Tiny 2420, an 8-bit processor with 2K of on-board flash memory), FPGA processors, ARM and RISC processors. Processor 530 is programmed such that, on power-up, it exercises register bit control of registers of image sensor 230, to cause image sensor 230 to rotate, invert or mirror the image stream, as necessary to compensate for the effect of the chosen prism. An exemplary image sensor 230 is the OmniVision 7690 sensor marketed by OmniVision Technologies Inc., which supports mirror, flip, scaling and windowing functions. A power regulator 520 may be provided in the event the voltage available from image presentation component 32 is not suitable for orientation processor 530. Power regulator 520 may scale the supply voltage provided by a conductor 522 up or down as required by the selection of the orientation processor 520 and provide said modified power via a conductor 524 to orientation processor 530.

It should be understood that the electrical components described above, or functions performed by them, may be provided in image presentation component 32. For example, image processing logic in image presentation component 32, configured to resize the image stream, may also be configured to invert, mirror or flip the image stream, thus the visualization instrument may not need an orientation processor in the wire harness or in the camera. Such processing logic may re-orient the image stream even if the image sensor is not capable of performing such re-orientation. Further, the wire bundle may be connected to the image sensor and the image processing logic may be configured to manage the registers of the image sensor to re-orient the image stream without using an orientation processor in the wire harness or the camera, when the image sensor is capable of performing such re-orientation. Further, a power regulator existing in image presentation component 32 may be modified to operate with different light sources, for example by modifying the feedback loop as described above, without a power regulator in the wire harness or the camera. The wire bundle may include an integrated circuit with an identifying code therein, which the image processing logic may read to determine how to configure the light source power and the image stream orientation. The image processing logic may then output the corresponding feedback loop and re-orientation signals to control operation of the camera.

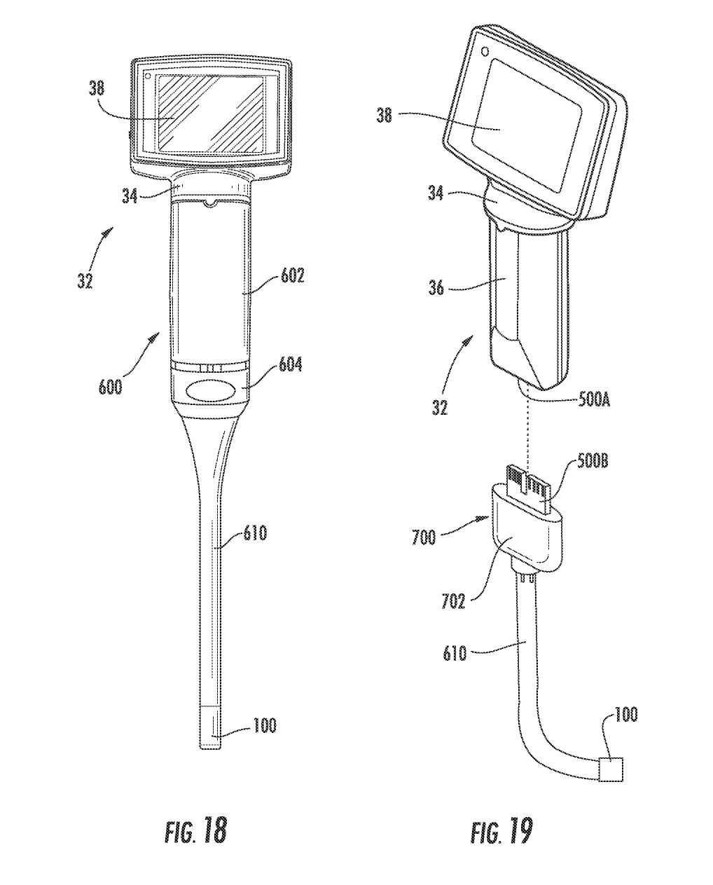

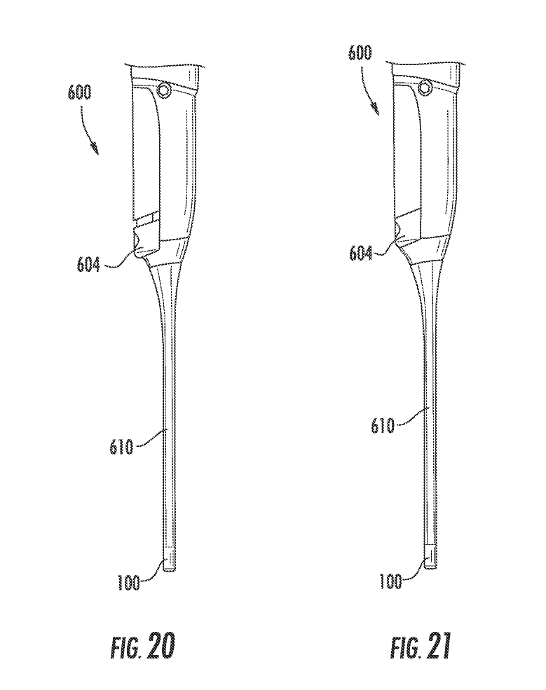

FIG. 18 is a plan view of an embodiment of image presentation component 32 connected to a blade adapter 600. Adapter 600 comprises a body 602, a sliding tab 604 and a wire harness 610 connecting body 602 to camera 100. Body 602 includes a proximal cavity (not shown) configured to receive battery housing 36. Body 602 is attached to battery housing 36 by sliding tab 604 from an unlocked position to a locked position. Camera 100 is permanently attached, and is part of, adapter 600. Camera 100 is communicatively coupled to presentation component 32 when presentation component 32 is inserted into the proximal cavity of body 602. FIG. 19 illustrates an embodiment of a detachable camera assembly 700 including a connector grip 702 and connector 500B coupled to wire harness 610. A female connector 500A (not shown) is located in image presentation component 32. When the handle cavity of blade 40 receives battery housing 36, wire harness 610 is received by the electronics pathway such that the camera view port is adjacent the blade view port. Any of the previously disclosed cameras may be used in place of camera 100.

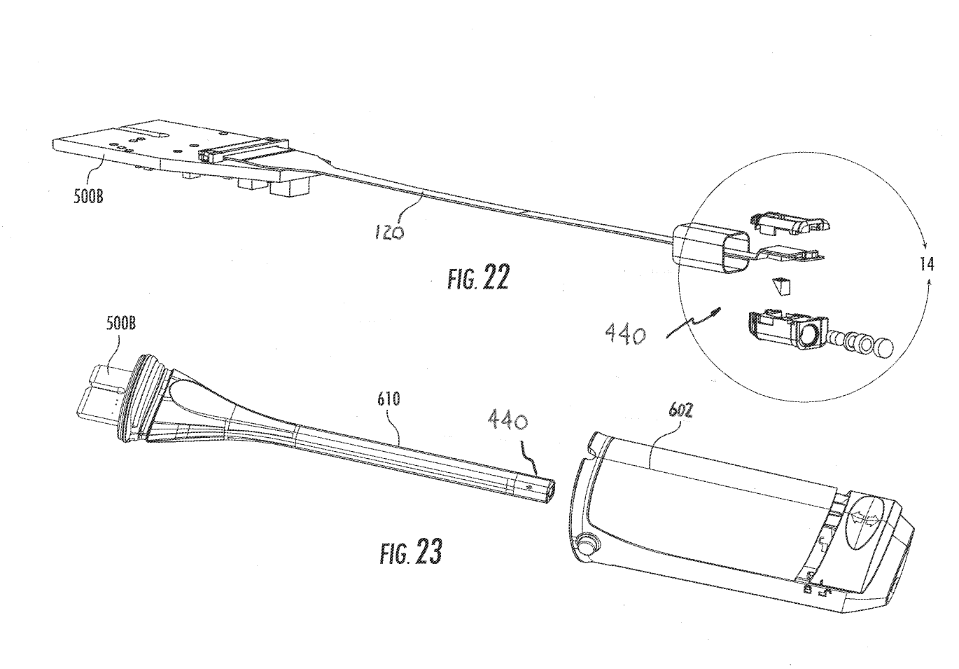

Referring again to blade adapter 600, FIGS. 20 and 21 illustrate, respectively, sliding tab 604 in the unlocked and locked positions. Once locked, blade adapter 600 remains attached to image presentation component 32. Wire harness 610 encloses wire bundle 120, which is permanently connected to camera 100. When body 602 receives battery housing 36, wire harness 610 is received by the electronics pathway such that the camera view port is adjacent the blade view port. Blade adapter 600 also comprises a connector (not shown) located in the proximal cavity suitable to electrically couple wire harness 610 to image presentation component 32.

FIGS. 22 and 23 are perspective views of another embodiment of an adapter, similar to adapter 600. In the present embodiment, the adapter includes adapter body 602, and wire harness 610 enclosing wire bundle 120, which is permanently connected to camera 440, previously described with reference to FIG. 14. Wire bundle 120, illustratively a flexible flat cable, extends from camera 440 to connector 500B, where it is attached to connector 500B with a mechanical brace. As shown, electronic components described with reference to FIGS. 15-17, such as power regulator 510, power regulator 520, and orientation processor 530, may be located on connector 500B. Removal of these components from the camera is desirable to achieve camera size reductions.

While the invention has been described as having exemplary designs, the present disclosure may be further modified within the spirit and scope of this disclosure. This application is therefore intended to cover any variations, uses, or adaptations of the disclosure using its general principles. Furthermore, this application is intended to cover such departures from the present disclosure as come within known or customary practice in the art to which this invention pertains.

1. A visualization instrument comprising:
an image presentation component including a display support housing coupling a battery housing and a display screen;
a blade including a handle portion integrally formed with an insertable portion, the handle portion comprising a proximal cavity configured to receive the battery housing, and the insertable portion comprising a medial wall, an elongate guide pathway on one side of the medial wall, and an electronics pathway on the other side of the medial wall;
a camera removably positionable in the electronics pathway and including:
a support housing including a camera view port, a prism cavity, and a lens cavity configured to receive one or more lenses;
a light source mounted on the support housing and adapted to illuminate structures in a target space;
an image sensor to generate a video stream, the image sensor configurable to change an orientation of the video stream;
a wire bundle permanently or removably affixed to the image presentation component and connected to the image sensor and the light source;
an optical train including the one or more lenses and a prism, the prism located adjacent the image sensor in the prism cavity and adhered to the image sensor, and the optical train sealed in the support housing; and
an assembly housing enclosing the support housing, the light source, the image sensor, and the optical train.

2. (canceled)

3. (canceled)

4. A visualization instrument as in claim 1, wherein the prism is a right-angle prism and the image sensor is perpendicular to the camera view port, further comprising a circuit configured to cause the image sensor to re-orient the video stream to compensate for the use of the prism, thereby compatibilizing the camera such that the image presentation component can also be used with a camera devoid of a prism.

5. A visualization instrument as in claim 4, wherein the circuit comprises an orientation processor mounted in one of the wire harness and the camera.

6. (canceled)

7. A visualization instrument as in claim 1, wherein the image sensor has a sensing surface, and the camera view port and the sensing surface are arranged at an angle of between about 30 and 90 degrees along a lens axis.

8. (canceled)

9. A visualization instrument as in claim 1, the camera further comprising an orientation processor connected to the image sensor by the wire bundle and configured to cause the image sensor to change an orientation of the video stream.

10. A visualization instrument as in claim 9, wherein the orientation is changed by inverting, mirroring or flipping the video stream.

11. A visualization instrument as in claim 9, the camera further comprising a power regulator configured to convert an input power to an output power and to supply the output power to the orientation processor.

12. A visualization instrument as in claim 11, wherein the power regulator is enclosed by the assembly housing.

13. A visualization instrument as in claim 1, further comprising orientation logic in the image presentation component configured to reorient the video stream for presentation with the display screen in an orientation that matches an orientation of the illuminated structures.

14. A visualization instrument as in claim 1, wherein the light source is positioned such that a projection of the light source overlaps the image sensor.

15. A visualization instrument as in claim 1, wherein the image sensor is positioned such that a projection of the camera view port overlaps the image sensor.

16. A visualization instrument as in claim 1, further comprising a blade adapter comprising the camera and a body including proximal cavity configured to receive the battery housing and having a connector therein configured to connect the camera to the presentation component when the presentation component is inserted into the proximal cavity of the body.

17. A method of making a visualization instrument, the method comprising:
electrically coupling an image sensor to a wire bundle, the image sensor configured to generate a video stream and configurable to change an orientation of the image video stream;
mounting a light source on a support housing;
inserting one or more lenses into a lens cavity of the support housing;
inserting a prism into a prism cavity of the support housing, the prism and the one or more lenses forming an optical train;
placing the image sensor adjacent the prism;
adhering the prism to the image sensor;
sealing the optical train in the support housing; and
enclosing the support housing with an assembly housing.

18. A method as in claim 17, wherein adhering the prism to the image sensor is performed before inserting the prism into the prism cavity.

19. A method as in claim 17, wherein sealing the optical train comprises adhering a distal lens of the optical train to the support housing.

20. A method as in claim 17, wherein sealing the optical train comprises adhering a lens cover to the support housing.

21. A method as in claim 17, further comprising sealing the light source in the support housing.

22. (canceled)

23. A method as in claim 17, wherein the support housing comprises a first support structure and a second support structure, further comprising positioning the wire bundle and the image sensor between the first support structure and the second support structure.

24. A method as in claim 17, wherein the prism is a right-angle prism, further comprising electrically coupling a circuit to the image sensor, the circuit configured to cause the image sensor to re-orient the video stream to compensate for the use of the prism, thereby compatibilizing the camera for use with an image presentation component operable with a camera devoid of a prism.

25. A method as in claim 24, the visualization instrument further comprising the image presentation component and a wire harness including a connector adapted to connect the wire harness to the image presentation component, the wire harness connected to the image sensor, further comprising mounting the circuit on one of the wire harness and the camera.

26. A method as in claim 24, the circuit comprising an orientation processor.

27. A method as in claim 26, further comprising mounting a power regulator on one of the wire harness and the camera, the power regulator adapted to power the orientation processor.
